(19) United States
(12) Patent Application Publication
(10) Pub. No.: US 2024/0407716 A1
(43) Pub. Date: Dec. 12, 2024

(54) ADHERENCE MONITORING SYSTEM AND METHOD

(71) Applicant: The Regents of the University of Michigan, Ann Arbor, MI (US)

(72) Inventors: Susan BROWN, Ann Arbor, MI (US); David T. BURKE, Ann Arbor, MI (US); Alanson SAMPLE, Ann Arbor, MI (US); Stephen M. CAIN, Morgantown, WV (US); Paula Anne NEWMAN-CASEY, Ann Arbor, MI (US)

(21) Appl. No.: 18/697,377

(22) PCT Filed: Sep. 30, 2022
(86) PCT No.: PCT/US22/45471

§ 371 Date: Mar. 29, 2024

Related U.S. Application Data
(60) Provisional application No. 63/251,242, filed on Oct. 1, 2021.

(51) Int. Cl. A61B 5/00; A61F 9/00

(52) U.S. Cl. CPC A61B 5/4833; A61F 9/0008

(57) ABSTRACT

An adherence monitoring system and method that can be used to determine instillation success, particularly with respect to eye drop instillation and other activities that involve bringing users hands to their heads. In one implementation, an eye drop adherence monitoring system comprises a processor and a sensor platform configured to attach to an eye drop container. The sensor platform includes one or more sensors configured to measure information relating to one or more instillation movements. The processor is configured to use the information relating to one or more instillation movements to determine instillation success.

### FIELD

This invention relates generally to medication monitoring, and more particularly, to methods and systems for monitoring eye drop usage.

### BACKGROUND

The World Health Organization has emphasized the importance of increasing the effectiveness of adherence interventions, and that doing so may have a far greater impact on the health of the population than any improvement in specific medical treatments. The National Institutes of Health (NIH), recognizing that behavior change is a key roadblock to improving health, instituted the Common Fund initiative to catalyze research in the Science of Behavior Change program. This NIH program acknowledges that behavior change can be exceptionally difficult for people to initiate and maintain. A key step in supporting patient autonomy and intrinsic motivation for health behavior changes is to measure the desired behavior. For example, in people with diabetes, glucose levels are measured-sometimes continuously with sensors—to enable informed decisions about food choices. Similarly, accurate quantification of adherence to glaucoma medications will enable individuals to improve self-regulation and self-management. Knowing whether a person (1) intended to take the medication by assessing the time/date that drops are dispensed, and (2) successfully instilled medication into the eye, will inform personally-tailored interventions to improve adherence. It is also desirable to understand sensorimotor and biomechanical deficits that impact the ability to successfully instill eye drops since additional support-assistive devices or exercises—can further improve eye drop success.

Despite the availability of effective treatments, glaucoma remains the leading cause of irreversible blindness among African Americans (and second overall) in the U.S. Three million Americans currently live with glaucoma, and it will rise to 7.3 million by 2050 as our population ages. Non-adherence to daily eye drop medications—the treatment for 89% of glaucoma patients—is a key modifiable driver of vision loss in glaucoma. People with lower glaucoma medication adherence are more likely to lose vision, to be of racial/ethnic minority background, and to have lower socioeconomic status. In current practice, patients are diagnosed with glaucoma and simply given a prescription: only 1 in 8 physicians teach patients how to use their eye drops. Glaucoma patients do not use the drops as scheduled at least 40% of the time and 20% of patients do not successfully instill the drop into their eyes. Glaucoma primarily affects older adults, a population for whom sensorimotor deficits of aging may impair successful medication instillation.

In order to reduce glaucomatous vision loss, it is advantageous to monitor medication use, quantify whether administered drops actually get into the eyes, communicate usage data to the patient's health care team, and coach patients on how to use their eye drop medications. Prior research of eye drop use behavior has been qualitative and observational. By strategically using the biomechanical and sensorimotor factors associated with eye drop instillation success, more effective, personalized intervention strategies to improve success in the use of eye drops can be developed.

High rates of poor adherence to effective eye drop medications are a key driver in glaucoma's persistence as a leading cause of irreversible blindness. Non-adherence to daily eye drop medications—the treatment for 89% of glaucoma patients—is a significant modifiable factor to improve vision outcomes for people with glaucoma. This disease primarily affects older adults (average age of onset 66 years), a population for whom successful glaucoma medication installation may be affected by sensorimotor deficits of aging.

In glaucoma, eye drop medication adherence has a multi-step definition: 1) obtaining the eye drop medication from the pharmacy: 2) accessing the medication at a scheduled time daily; and 3) successfully instilling the medication into the eye. Failure at any of these steps can lead to poor adherence and poor vision outcomes. Most previous work assessing glaucoma medication adherence has either used self-reported adherence, which has poor reliability: pharmacy claims records, which assess only Step 1: or electronic “adherence monitors” developed for pill medications, which assess only whether the eye drop bottle was removed from the monitoring container (Step 2). No known objective eye drop adherence monitoring technology rigorously assesses Step 3, whether the medication was successfully instilled into the eye.

It is thus advantageous to have an eye drop adherence monitoring system that can provide high-quality quantitative data on eye drop instillation to patients and their health care team that has been rigorously evaluated and has been demonstrated to generate reproducible data. Detailed individual adherence behavior data that can be communicated between providers and patients are helpful in creating clear goals for glaucoma self-management. Accordingly, an adherence system should be designed to provide glaucoma self-management support for all patients including those with lower incomes, minority backgrounds, and those of older age, and in some embodiments, with no requirement for costly home computers, broadband internet, or smartphones. For example, although 91% of Americans over age 65 have a cellphone, only 52% use a smartphone.

It is estimated that about half of patients do not adhere to their glaucoma medications: poor adherence is associated with vision loss. In one randomized controlled clinical trial comparing a single coaching session to standard care, standard care group participants missed 38% of prescribed glaucoma medication doses. Prior work demonstrated that worse self-reported medication adherence predicted vision loss from glaucoma: study participants with a missed dose of medication at up to one-third of study visits had twice the vision loss over nine years compared to participants who reported perfect adherence.

Among patients who successfully obtain their medication, 20% cannot instill an eye drop into the eye. Further, among those with advanced vision loss from glaucoma, 30% cannot successfully instill a drop. A recent review of the eye drop instillation technique literature found that 80% of patients contaminate their eye drop bottle when instilling drops and 60% do not instill the appropriate volume. The gold standard assessment of eye drop instillation in the 15 studies reviewed was having an observer grade video-recorded eye drop instillation. Currently, no known systems quantitatively assess eye drop instillation technique and success.

Sensorimotor deficits of aging likely impact glaucoma medication instillation success. Hand function is critical for daily activities requiring precise sensorimotor control, but declines in older adults as a result of age-related muscle fiber remodeling and loss. Older adults have reduced somatosensory receptor sensitivity, and structural alterations in movement-related brain structures. These changes lead to impairments in proprioception, fine grasp force control-which includes hand steadiness, tactile discrimination (the ability to identify object characteristics based on touch), and hand function including the dexterity needed for bimanual and limb-posture coordination. A recent survey to assess upper extremity disability demonstrated an effect of disability on the volume of medication dispensed, but did not demonstrate an effect on the ability to successfully instill an eye drop. Precise functional assessments of sensorimotor ability will more accurately characterize age-related deficits associated with instilling eye drops.

Racial and socioeconomic disparities in medication adherence can also contribute to disparities in glaucoma outcomes. African Americans are 1) at increased risk for glaucoma. 2) at increased risk of blindness due to glaucoma, and 3) are less likely to adhere to treatment. Those with low income are also more likely to have glaucoma. In a pilot study of a glaucoma coaching program, a household net income of less than $25,000 increased the risk of poor adherence, with income explaining 22% of adherence variation. Clearly, strategies to improve adherence and self-management must be broadly inclusive and should not exclusively rely on expensive technologies that many patients do not have (e.g., broadband internet or smartphones).

Prior work has relied on subjective measures of eye drop instillation success, which are often not quantifiable. This technical problem can hinder treatment outcomes, as the subjective measures (e.g., watching a video of the patient) can be difficult to implement and execute. Thus, there is a technical advantage that can improve treatment outcomes if a system is developed to quantify and monitor successful eye drop utilization and instillation. The existing standard in studies assessing eye drop technique has been video-recorded instillation with a masked observer using a check-list to grade: 1) whether the eye drop entered the eye and 2) whether the tip of the bottle was contaminated by touching the skin or ocular surface. In contrast to subjective observation, biomechanical analyses could instead be used to provide objective measures of human movement (e.g., joint angles, postures, movement speeds) to define and quantify technique.

Thus, the technical advantages discussed herein can have a large impact: three million people in the U.S. live with glaucoma; and with the aging of the population, it will be 7 million by 2050. Clearly, new strategies and technologies must be employed to improve glaucoma management. Approaches to improving glaucoma care will require tools such as those described herein to: 1) quantify whether eye drops were dispensed on schedule, 2) quantify whether they were successfully instilled into the eye, and 3) communicate the data to the patient and their health care team to facilitate improvement. Objective, quantitative medication use data will inform personalized, scalable approaches to eye drop instillation coaching and will empower tailored aids to improve instillation techniques. Researchers can use the systems and methods herein as a new gold standard in measuring eye drop medication adherence. Based on the glaucoma adherence model, similar strategies may improve medication adherence in other eye conditions, such as following cataract surgery-currently the most commonly performed elective surgery in the USA (about 22 million people annually).

Typical “gold standard” medication adherence monitors are designed to assess adherence to oral medications. Currently, health professionals also measure glaucoma medication adherence with these monitors. Removing the cap of the monitor is considered a proxy for the patient accessing the eye drop bottle and putting medication into the eye. New monitor devices for eye drop medications have recently been developed: however, these are not commercially available and cannot assess eye drop instillation success. Moreover, to close the communication loop between patient and clinician, a monitoring system should report use-events and metrics in approximately real-time.

### SUMMARY

In accordance with one embodiment, there is provided an eye drop adherence monitoring system comprising a processor and a sensor platform configured to attach to an eye drop container. The sensor platform includes one or more sensors configured to measure information relating to one or more instillation movements. The processor is configured to use the information relating to one or more instillation movements to determine instillation success

In various embodiments, the processor and the sensor platform are integrated on a sleeve.

In various embodiments, the processor and the sensor platform are integrated on a sticker.

In various embodiments, the processor is associated with a base station.

In various embodiments, the processor is part of a microcontroller that facilitates wireless communication to a base station.

In various embodiments, the information relating to one or more instillation movements includes biomechanical data relating to a posture, a limb position, and/or a dynamic movement of a user.

In various embodiments, biomechanical data relating to a posture includes a measurement of thorax tilt, a measurement of head tilt, a measurement of a neck flexion-extension angle and/or a measurement of a neck lateral flexion angle.

In various embodiments, biomechanical data relating to a limb position includes a measurement of an elbow flexion-extension angle, a measurement of an elbow supination-pronation angle, a measurement of an angle of elevation for a shoulder, a measurement of a plane of elevation of the shoulder, and a measurement of a wrist height relative to the shoulder.

In various embodiments, the information relating to one or more instillation movements includes sensorimotor data relating to a proprioception quantification, a fine grasp force control, a tactile discrimination, and/or a hand function.

In various embodiments, the information relating to one or more instillation movements includes a duration of an instillation pause, a steadiness of the eye drop container during instillation, and a smoothness of a position trajectory of the eye drop container.

In various embodiments, the one or more sensors includes an inertial measurement unit (IMU), a capacitive sensor, and a magnetic switch, and ultrasonic transducer.

In various embodiments, the processor is configured to calculate a position trajectory for the eye drop container based on an orientation, a velocity, and a position of the eye drop container.

In various embodiments, a radio communication unit is integrated with the sensor platform.

In various embodiments, the radio communication unit is a backscatter radio communication unit.

In accordance with another embodiment, there is provided an adherence monitoring system, comprising a processor and a sensor platform configured to attach to a container. The sensor platform includes one or more sensors configured to measure information relating to one or more instillation movements. The processor is configured to use the information relating to one or more instillation movements to determine instillation success. The information relating to one or more instillation movements includes biomechanical data relating to a posture, a limb position, and/or a dynamic movement of a user. The information relating to one or more instillation movements includes sensorimotor data or a duration of an instillation pause, a steadiness of the container during instillation, and a smoothness of a position trajectory of the container.

In various embodiments, the biomechanical data relating to the posture includes a measurement of thorax tilt, a measurement of head tilt, a measurement of a neck flexion-extension angle and/or a measurement of a neck lateral flexion angle.

In various embodiments, the biomechanical data relating to the limb position includes a measurement of an elbow flexion-extension angle, a measurement of an elbow supination-pronation angle, a measurement of an angle of elevation for a shoulder, a measurement of a plane of elevation of the shoulder, and a measurement of a wrist height relative to the shoulder.

In various embodiments, the sensorimotor data relates to a proprioception quantification, a fine grasp force control, a tactile discrimination, and/or a hand function.

In accordance with another embodiment, there is provided a method of eye drop adherence monitoring, comprising the steps of: obtaining information relating to one or more instillation movements from a sensor platform attached to an eye drop container; and determining instillation success from the information relating to one or more instillation movements.

In various embodiments, the information relating to one or more instillation movements includes biomechanical data relating to a posture, a limb position, and/or a dynamic movement of a user, and wherein the information relating to one or more instillation movements includes sensorimotor data or a duration of an instillation pause, a steadiness of the container during instillation, and a smoothness of a position trajectory of the container.

It is contemplated that any number of the individual features of the above-described embodiments and of any other embodiments depicted in the drawings or description below can be combined in any combination to define an invention, except where features are incompatible.

### DRAWINGS

Example embodiments will hereinafter be described in conjunction with the appended drawings, wherein like designations denote like elements, and wherein:

FIG. 1 is a schematic representation of an eye drop adherence monitoring system in accordance with one embodiment;

FIG. 2 is an image of a sleeve for a sensor platform for an eye drop adherence monitoring system:

FIG. 3 is an image of a sticker for a sensor platform for the eye drop adherence monitoring system of FIG. 1:

FIG. 4 is a schematic block diagram of the system of FIG. 1;

FIG. 5 schematically illustrates various instillation movements and biomechanical data measurements that can be used with the eye drop adherence monitoring system:

FIG. 6 is a graph showing biomechanical data in the form of a wrist height relative to the shoulder of the user of FIG. 5; and

FIG. 7 is a graph showing biomechanical data in the form of an elbow flexion-extension angle of the elbow of the user of FIG. 5; and

FIG. 8 is a graph showing a position trajectory for the eye drop container of the user illustrated in FIG. 5.

### DESCRIPTION

An eye drop adherence monitoring system and method is described herein that is designed to determine instillation success, by predicting whether an eye drop successfully entered a user's eye, advantageously with an over 80% success rate. This information can be transmitted to a medical professional in real-time or almost real-time to improve treatment outcomes. Strategically configured sensors located on the eye drop container, along with wireless communication capabilities, provide information relating to eye drop instillation to a medical professional in a relatively easy to implement fashion. This allows for medical professionals to improve adherence rates, thereby providing better treatment outcomes. This is especially pronounced with respect to glaucoma treatments. However, while the focus is on glaucoma treatments, the systems and methods herein can be used to address any other condition needing eye drop medications, and is not limited to treating glaucoma. Additionally, the present disclosure relates to instillation success, which may be particularly applicable to eye drops, as well as other hygiene activities, eating and drinking, adjusting hearing aids, etc. Accordingly, instillation may relate to other activities requiring motion of the upper extremity that brings the hand to the head.

The systems and methods herein address five critical issues raised by the National Institute on Aging's Strategic Directions for Research, the NIH Adherence Research Network, the International Agency for the Prevention of Blindness, and the International Council for Ophthalmology: 1) poor glaucoma medication adherence rates: 2) high rates of inability to successfully instill eye drops among those obtaining glaucoma medication: 3) sensorimotor changes of aging and the potential impact on the ability to instill eye drops: 4) social and economic disparities in both glaucoma medication adherence and glaucoma outcomes; and 5) scalable strategies to quantify successful medication use and provide personalized support for patients from diverse backgrounds to improve glaucoma self-management and outcomes.

The devastating vision loss that ensues from glaucoma has a simple solution for the majority of cases—taking eye drop medications accurately and on schedule. Objective, real-time data quantifying patients' eye drop use will enable individualized strategies to improve glaucoma medication adherence. Currently, no known low-cost, deployable strategies exist that: (1) identify when an eye drop medication was dispensed, (2) determine the probability of successful medication instillation, and (3) share medication adherence data between patients and their clinicians. The eye drop adherence monitoring system described herein can help remedy this market deficiency.

In contrast to subjective observation, biomechanical analyses could instead be used to provide objective measures of human movement (e.g. joint angles, postures, movement speeds, etc.) to define and quantify technique. For example, inertial measurement units (IMUs: wearable sensors that measure motion) quantify human performance and technique across a wide range of activities and are routinely used to provide feedback to athletes. Data from a single IMU can be used to understand the movement trajectory (position) and movement speed of a body segment or object, while data from multiple IMUs can estimate joint angles. Body-worn IMUs synchronized with measurements from the eye drop bottle monitor can accordingly provide objective measures of eye drop instillation.

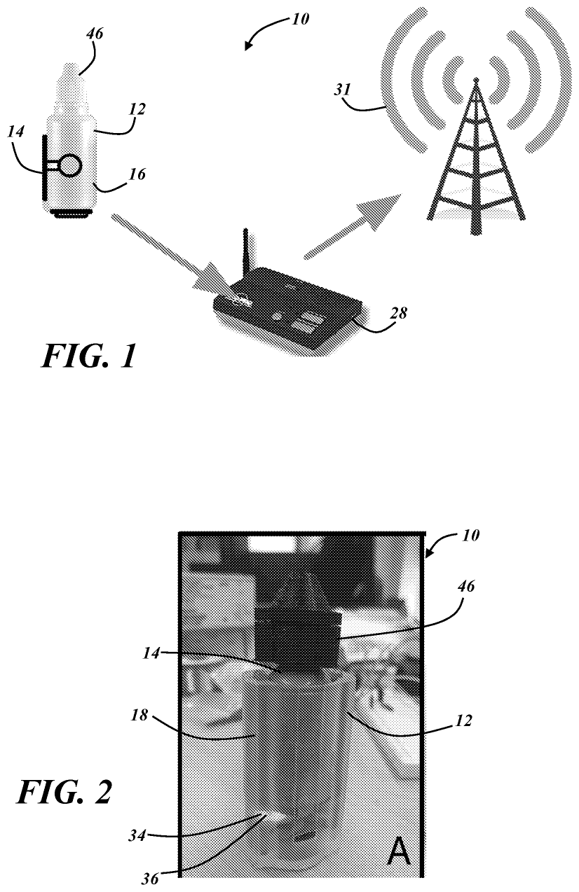

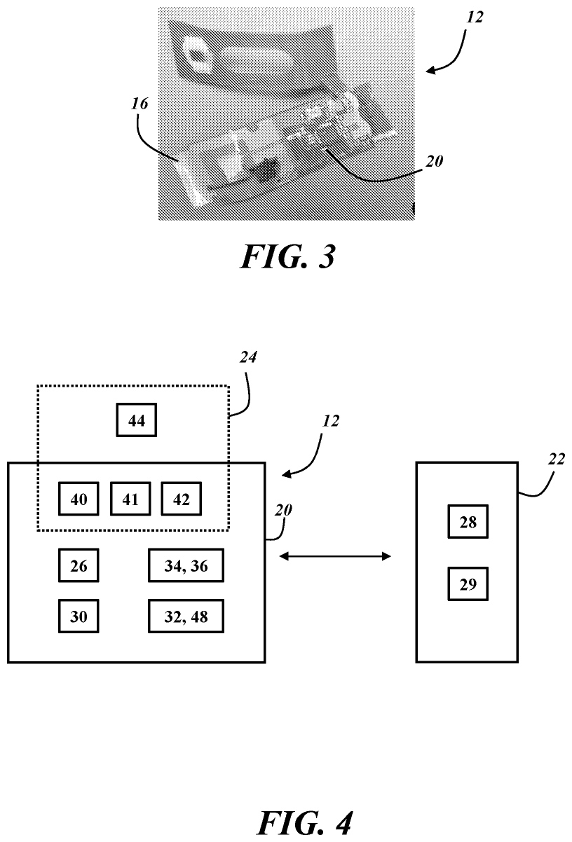

FIGS. 1-4 illustrate an eye drop adherence monitoring system 10. With particular reference to FIG. 1, a monitoring device 12 is situated on an eye drop bottle or container 14 to monitor eye drop usage and instillation success. The monitoring device 12 can be in the form of a sticker 16, such as that shown in the examples of FIGS. 1 and 3, or a sleeve 18, such as that shown in the example of FIG. 2. The sticker 16 and sleeve 18 provide for intuitive attachment to the eye drop bottle 14, and it should be understood that teachings relating to the sticker 16 embodiment are applicable to the sleeve 18 embodiment and vice versa (unless features are otherwise incompatible). The sticker 16 and/or sleeve 18 may be made of silicone according to one embodiment, and may be configured to be disposable (e.g., once the medication is done, the monitoring device 12 can be thrown away with the container 14). In some embodiments, the sticker 16 and/or sleeve 18 includes an advanced flexible printed circuit board that easily wraps around the bottle 14, avoiding significant modification. The sticker 16 and the sleeve 18 are just examples, however, and the monitoring device 12 can take a number of different forms, advantageously forms that allow for convenient attachment to the eye drop bottle 14. Advantageously, the monitoring device 12 is a low power, portable Internet of Things (IoT) system that can be used daily and is easily joined with the eye drop container 14.

With reference to the schematic of FIG. 4, the eye drop adherence monitoring system 10 includes internal electronic components 20 to the monitoring device 12, as well as external electronic components 22 for the eye drop adherence monitoring system 10 (i.e., the components remote from the container 14). The internal electronic components 20 include a sensor platform 24 and a processor 26. Various subcomponents of the sensor platform 24 and/or the processor 26 may be integrated with the sticker 16 such that they are located on-bottle, or may be distributed to other on-bottle locations, or they may be remote from the bottle (e.g., associated with an external base station 28), to cite a few operational arrangements. Locating them on-bottle can help in some instances improve data acquisition. For example, locating one or more sensors on the side of the container 14 may improve data acquisition as compared to other on-bottle locations (e.g., on the bottom) as they are located closer to the typical grasping location. Further, various subcomponents that are separately illustrated may be combined into one or more other subcomponents (e.g., one or more sensor may be a sensing unit with a dedicated processor). The illustrated schematic is only one example operational arrangement. Other components of the eye drop adherence monitoring system 10 may include memory 30, a wireless communication unit 32, and a human machine interface 34, which in this embodiment, includes a small light source 36.

The sensor platform 24 includes one or more sensors which are configured to measure information relating to one or more instillation movements. In the illustrated embodiment, there is a movement sensor 40, a pressure senor 42, and a cap sensor 44. As detailed below, however, more or less sensors may be used than what is schematically illustrated herein. The sensor platform 24 and sensors 40-44 are configured to provide information relating to when an eye drop medication is dispensed, the probability of successful medication instillation, along with adherence data in general. This allows for medication adherence data to be more easily shared between patients and their clinicians. The sensor platform 24 advantageously includes the minimal number of sensors needed to sufficiently determine the probability of instillation success, which results in a more simplified, low-cost structure that can be rapidly scaled. Additionally, such a design process can help ensure the system 10 will be user-friendly for people with minimal communication and computational infrastructure or experience. Moreover, the sensor platform 24 and one or more sensors 40-44 can be configured to provide objective, real-time data that quantifies patients' eye drop use. This can enable individualized strategies to improve glaucoma medication adherence through the use of a lower-cost, more deployable strategy.

The movement sensor 40 is preferably one or more inertial measurement units (IMUs). The sensor 40 can accordingly be used to obtain movement or inertial information concerning the eye drop container 14, such as container speed, acceleration, yaw (and yaw rate), pitch, roll, and various other attributes of the container concerning its movement as measured locally through use of on-bottle sensors. The movement sensor 40 can be coupled to various other electronics 20, such as the processor 26. Movement sensor data can be obtained and sent to the processor 26 and/or wireless communications unit 32. While the movement sensor 40 in the illustrated embodiment is an IMU, it is possible for other accelerometers, gyroscope sensors, or other inertial sensors to be used. Additionally, the movement sensor 40 may be a more simple speed or velocity sensor, or could include other sensors, such as separate angular position sensors or yaw rate sensors, to cite a few examples.

The IMU movement sensor 40 can be a microelectromechanical system (e.g., a MEMS sensor) or accelerometer that obtains inertial information relating to a position trajectory for the eye drop container 14. Such inertial information may include an orientation, a velocity, and/or a position of the eye drop container 14. Additionally, shaking behavior of the patient can be mapped. The IMU 40 can be a multi-axis accelerometer that can measure acceleration or inertial force along a plurality of axes. In an advantageous embodiment, the IMU sensors 40 measure the motion of the container 14 (linear acceleration and angular velocity) with high resolution as the patient delivers the eye drop onto the eye. This can help provide biomechanical movement data that maps eye drop instillation technique in three dimensions, over time. Other embodiments may employ single-axis accelerometers or a combination of single- and multi-axis accelerometers. Other types of sensors can be used, including other accelerometers, gyroscope sensors, and/or other inertial sensors that are known or that may become known in the art. In one embodiment, an ultrasonic transducer 41 is included as a sensor to help determine fluid levels in the container 14.

The pressure sensor 42 can be used to provide information relating to instillation success, such as fine grasp force control, tactile discrimination, and/or hand function. In an advantageous embodiment, the pressure sensor 42 is a capacitive sensor that can also measure the fluid sensor in the container 14. In one embodiment, two plates made from copper tape are formed into a cylinder or semi-cylinder shape to conform to the outside of the container 14. The two plates can act as capacitors in parallel, with the top capacitor measuring the empty volume of the bottle (filled with air) and the bottom capacitor measuring the modification. A capacitance to digital convertor can measure the capacitance across the volume of the container 14. In other embodiments, the pressure sensor 42 can be a MEMS force sensor or other operable force sensor that is configured to measure sensorimotor data.

The cap sensor 44 is used to provide information relating to the open or closed status of the cap 46 of the eye drop container 14. In one embodiment, the cap sensor 44 is a magnetic switch comprised of two reed switches and magnets embedded in a 3D printed cap. This cap can be used to replace the original container cap without modifying the medication container 14 functionality. The cap sensor 44, as well as the pressure sensor 42, are optional and can help provide corroborating data to the determination of instillation success. In some embodiments, the cap sensor 44 may be used as a trigger to determine when data from the other sensor(s) should be sent to the base station 28.

The processor 26 is advantageously a microcontroller configured to receive information from the sensor platform 24. Sensor information and data can be stored in memory 30 and used by the processor to determine instillation success. Processor 26 can be any type of device or set of devices capable of processing electronic instructions including microprocessors, microcontrollers, host processors, controllers, and application specific integrated circuits (ASICs). It can be a dedicated processor used only for one or more of the sensors 40-44, or it can be shared with other system 10 components (e.g., the wireless communication unit 32 and/or HMI 34), to cite a few operational arrangements. Processor 26 executes various types of digitally-stored instructions, such as software or firmware programs stored in memory 30, which enable the device 12 to provide a wide variety of information. Memory 30 may be a temporary powered memory, any non-transitory computer-readable medium, or other type of memory. For example, the memory can be any of a number of different types of RAM (random-access memory, including various types of dynamic RAM (DRAM) and static RAM (SRAM)), ROM (read-only memory), solid-state drives (SSDs) (including other solid-state storage such as solid state hybrid drives (SSHDs)), etc.

Wireless communications unit 32 is capable of communicating data to a base station 28, which may be its own stand-alone device or may be a user's mobile device 29, or both. The base station 28 and/or a user's mobile device 29 can then communicate, using any operational means such as via a cellular network 31, to the user's health care provider. In one advantageous embodiment, the wireless communications unit 32 is or includes a backscatter radio unit 48. The backscatter radio unit 48 uses 100-fold less power consumption, and can reduce system complexity by eliminating traditional radio components (e.g., power amplifiers, RF mixers, active filters). Additionally, to keep operational costs minimal, existing cellular data plans specifically designed for IoT applications can be used. The wireless communications unit 32 sends data to the base station 28 to be stored, analyzed, and transmitted back to the health care team. Adherence summaries can be reported back to participants by automated text messages or phone calls in some embodiments.

While collecting on-bottle sensor data using RF backscatter is preferable, other communication forms can be used with the system 10. In some embodiments, the wireless communications unit 32 can be configured to communicate wirelessly according to one or more short-range wireless communications (SRWC) such as any of the Wi-Fi™ WiMAX™, Wi-Fi Direct™, other IEEE 802.11 protocols, ZigBee™, Bluetooth™ Bluetooth™ Low Energy (BLE), or near field communication (NFC), to cite some examples. As used herein, Bluetooth™ refers to any of the Bluetooth™ technologies, such as Bluetooth Low Energy™ (BLE), Bluetooth™ 4.1, Bluetooth™ 4.2, Bluetooth™ 5.0, and other Bluetooth™ technologies that may be developed. As used herein, Wi-Fi™ or Wi-Fi™ technology refers to any of the Wi-Fi™ technologies, such as IEEE 802.11b/g/n/ac or any other IEEE 802.11 technology. In some embodiments, the wireless communications unit 32 is an integrated component of the microcontroller/processor 26. Other computational arrangements and configurations are certainly possible.

The monitoring device 12 may also include an HMI 34 such as a small LED light 36. Other HMI forms are certainly possible, such as a haptic feedback device or a device to provide an auditory cue to a user. The HMI 34 can be used to indicate that instillation was likely successful. For example, if the data indicates that the drop was successfully instilled into the user's eye, the light 36 may change color (e.g., blue to green). Also, the HMI 34 may be used to indicate that instillation was likely not successful. For example, if the data indicates that the drop was not successfully instilled into the user's eye, the light 36 may change color (e.g., from blue to red). This feature is optional, and other forms may be used to provide feedback to the user, such as automated text messages as described above.

The on-board processor 26 and/or processor 33 for the base station 28 can run real-time or almost real-time classification algorithms that detect use-events with 94% accuracy. Use-events and fluid levels can be transmitted to a nearby smartphone via Bluetooth and, subsequently, sent to the health care provider via Wi-Fi. The eye drop bottle monitor 12 can provide cues to patients using on-device indicator LEDs 36 or automated reminders (SMS or phone).

While typical systems are designed to determined when a drop is administered out of the container 14, no known systems determining if instillation was successful (i.e., whether the drop actually entered the patient's eye) with sufficient accuracy to impact patient outcomes. The EAMS 10 is designed to determine if instillation was successful, by estimating the likelihood of instillation success given information received from the sensor platform 24. The following information and parameters detailed below can be used to implement a system 10 that advantageously predicts successful instillation by 80% or more. While it is possible to use a prediction that is somewhat less than 80%, or greater than 80%, it is believed that this threshold can be used to sufficiently determine instillation success and adequately improve patient adherence to treatment regimens.

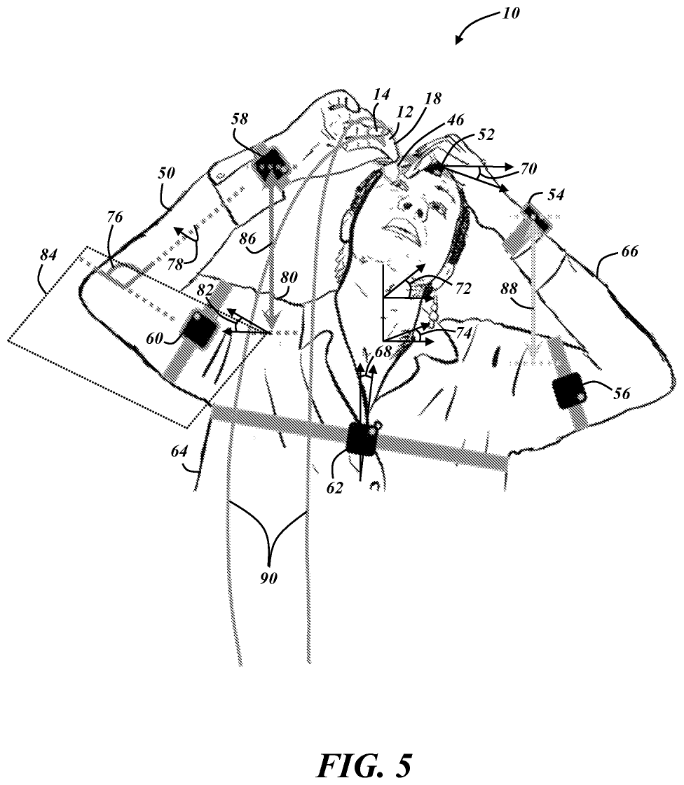

FIG. 5 illustrates an example design for setting up and configuring various parameters of the EAMS 10. A user 50 is equipped with wearable sensors (e.g., IMUs), such as a head sensor 52, a left lower arm sensor 54, a left upper arm sensor 56, a right lower arm sensor 58, a right upper arm sensor 60, and a torso sensor 62. These sensors, along with the sensor platform 24, can be used to obtain information relating to instillation movements to determine instillation success. In one embodiment, the information relating to one or more instillation movements includes biomechanical data relating to a posture, a limb position, and/or a dynamic movement of the user 50. For example, during instillation, a posture 64 as sensed by torso sensor 62, a limb position 66 as sensed by one or more arm sensors 54, 56, and a dynamic movement from one or more of the sensors could be used as biomechanical data. In some embodiments, biomechanical data relating to the posture 64 includes a measurement of thorax tilt 68, a measurement of head tilt 70, a measurement of a neck flexion-extension angle 72, and a measurement of a neck lateral flexion angle 74. In some embodiments, biomechanical data relating to the limb position 66 includes a measurement of an elbow flexion-extension angle 76, a measurement of an elbow supination-pronation angle 78, a shoulder 80 angle of elevation 82, a plane of elevation 84 for the shoulder 80, and a wrist height 86, 88 relative to the shoulder (such limb position data being taken from one or both arms, advantageously the arm holding container 14 during the instillation attempt). This set of biomechanical data described herein can help create a more robust movement profile to help better predict instillation success.

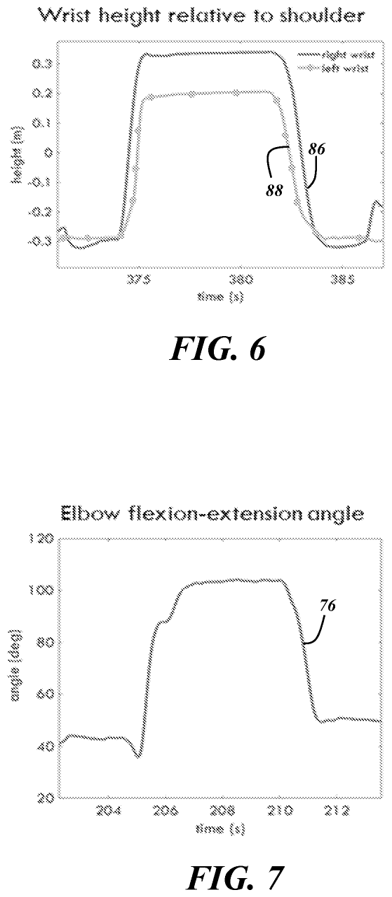

With a subset of one or more measurements described above obtained during instillation attempts, a profile can be developed that determines instillation success with a prediction having greater than 80% accuracy, advantageously. For example, FIG. 6 shows the wrist height relative to shoulder for the right and left wrists 86, 88, respectively, during an instillation attempt. Similarly, FIG. 7 shows an example graph of the elbow flexion-extension angle 76 taken during an instillation attempt. Anomalies and trends in these biomechanical data patterns can help determine instillation success, as detailed further below.

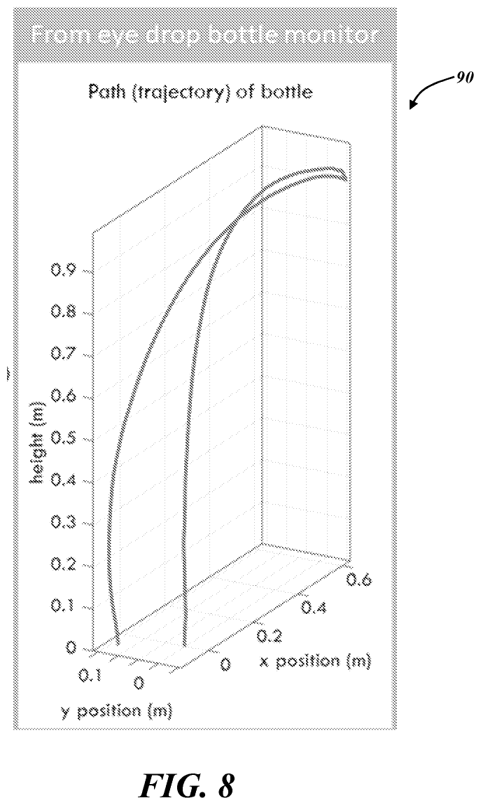

In some embodiments, as illustrated in FIGS. 5 and 8, the monitor 12 is configured to calculate a position trajectory 90 for the container 14. This may be accomplished, for example, with the IMU movement sensor 40 of the sensor platform 24, as described above. The position trajectory 90 can be calculated, in some embodiments, based on an orientation, a velocity, and a position of the eye drop container 14. This position trajectory 90 can help provide information relating to a duration of an instillation pause and a steadiness of the eye drop container 14 during instillation. The smoothness of the position trajectory 90, along with the duration of instillation paused and steadiness of the container can be used to help predict and determine instillation success. Additionally, information relating to one or more instillation movements can include sensorimotor data relating to a proprioception quantification, a fine grasp force control, a tactile discrimination, and/or a hand function, as detailed further below.

In some embodiments, it is desirable to quantify the biomechanics of eye drop instillation and identify biomechanical and sensorimotor factors that predict successful instillation among older adults. Biomechanical data can be collected using 1) multiple wearable sensors on the head/body/limbs as shown and described above and 2) an eye drop monitor 12 to obtain measures of posture, limb position, and dynamic movement. Sensorimotor data can be collected using performance tests to quantify proprioception (sense of self-movement and body position), fine force control, tactile discrimination, and hand function. Participant data (n=100, for example) can inform algorithms using machine learning to: 1) identify the biomechanical and sensorimotor factors that predict instillation success and 2) identify the fewest sensors necessary to predict instillation success with at least 80% accuracy.

The EAMS 10 can be created utilizing a user-centered design. The EAMS 10 will consist of a low-power, portable, on-bottle sensor platform 24 linked to a base station 28 and/or mobile device 29 that communicates the data via a cellular network to 1) identify when eye drop medication was dispensed, 2) determine the probability of successful medication instillation, and 3) share medication use and instillation success data between patients and providers. EAMS engineering fidelity should be assessed by, for example, collecting qualitative feedback on the EAMS to understand how to improve the design to make it user-friendly by interviewing a purposive sample of older adults from 1) racial/ethnic minority backgrounds who do not routinely use a computer/smartphone and 2) those with glaucoma.

In some embodiments, the EAMS 10 can be used clinically to improve the effectiveness of glaucoma care in preventing vision loss. It can be used to assess adherence to eye drop medications in both the clinical and research settings. New knowledge about the biomechanics of eye drop instillation and the impact of the sensorimotor deficits of aging on eye drop medication use can be obtained. This deeper understanding will enable patient-centered approaches to self-management support and aid in closing outcomes disparities in glaucoma—and every other condition needing eye drop medications. Using sensitive assessments of proprioception, fine force control, and tactile discrimination, the tools to understand how the aging process impacts the ability to instill eye drops can be improved. In the EAMS 10, use of backscatter radio communication technology will reduce power consumption, thereby requiring less-frequent re-charging.

Investigative work should use high-fidelity sensors, such as those illustrated in FIGS. 4 and 5, to measure patient motion, together with an eye drop bottle sensor platform 24 to measure bottle 14 motion. The sensors will record body postures and movements during instillation, allowing quantification of the biomechanics of eye drop use. For each user, sensorimotor abilities can be quantified for proprioception, fine grasp force control, tactile discrimination, and hand function, using validated performance-based tests and novel instruments. After obtaining quantitative information on eye drop instillation performance, machine learning can be used to identify the minimal set of biomechanical and sensorimotor factors that most accurately predict eye drop instillation success. This minimal set of factors can be integrated into the EAMS 10 with three components: 1) an on-bottle sensor 40 to measure spatial movement, when an eye drop is dispensed, and calculate the probability of successful instillation, 2) a base station 28 to collect data from the sensor and communicate date, time, and instillation probability for each use-event to the health care team, and 3) a provider interface to record and track patient use-events, capture adherence trends, and facilitate communication with the patient. The design of the EAMS 10 can be iteratively informed in some embodiments by interviewing glaucoma patients and older African-American adults who do not use a computer or smartphone daily for feedback about device usability.

Glaucoma prevalence increases four-fold between ages 40-79; meanwhile, the sensorimotor function necessary to instill medication eye drops decreases with aging. Hand sensorimotor skills were examined in 13 young (mean age: 20.0 years) and 13 older adults (mean age: 72.2 years) using two novel assessment tools. During tasks requiring fine grasp force control (similar to what is needed to instill eye drops), in the older group, fluctuations in force production increased by 20% in the dominant-hand and 80% in the non-dominant hand (p<0.01), demonstrating that older adults have difficulty maintaining smooth fine grasp force control. Discrimination of different tactile patterns was also impaired in the older group, with significant increases in pattern identification time regardless of hand (p<0.01) as well as decreases in accuracy (p<0.01). In contrast, maximum grip strength and monofilament detection, commonly used to assess hand function and tactile discrimination in clinical settings, did not differ between age groups. This underscores that current “gold standard” testing is not sufficiently sensitive to age-related changes. However, fine grasp force control and tactile discrimination are both associated with the age-related sensorimotor changes that are likely to impact eye drop instillation. Accordingly, this information can be used by the EAMS 10 to determine instillation success.

It has been demonstrated that personalized health coaching, coupled with adherence behavior feedback and reminders to glaucoma patients with poor adherence, significantly impacts medication adherence, improving from 59.9% (+18.5) at baseline to 81.3% (+17.6) after a 7-month intervention (p<0.0001). In one embodiment, the intervention uses a web-based tool that tailors health education and coaching to: type of glaucoma, test results, doctor's recommendations, barriers to use, and adherence level. To date, health coaching has focused on motivation to integrate eye drops into daily routines. With quantifying how each user instills their eye drops, we can personalize clinician's advice to the user's physical technique at the level of how, for example, a golf coach might inform a player's swing. This is a significant knowledge gap, because, in recent interventions, 25% of patients were unable to successfully instill eye drops.

As described earlier with respect to FIG. 5, a study team member can secure wireless IMU sensors to the participant's right/left wrists, right/left upper arms, head, and thorax using elastic hook and loop straps (e.g., six sensors). After donning the sensors, participants can perform a series of functional calibration movements and postures. The movements define sensor-to-body-segment alignments, which is advantageous for the accurate calculation of joint angles. After completing the functional calibration movements and poses, participants can instill artificial tear eye drops into both eyes, four times in each eye. An artificial tear eye drop bottle 14 will be placed inside the monitor 12 to obtain quantitative data on each instillation event. Participants will not be given guidance on instillation, as the goal is to capture a variety of self-selected instillation techniques. After the first instillation, the participants can be asked to instill eye drops in three common postures, four times per eye: 1) sitting in front of a mirror, 2) standing in front of a mirror; and 3) lying down supine. Information can be collected with respect to on bottle movement (bottle monitor IMU sensor 40) and bottle squeeze (bottle monitor 12, capacitive sensor 42). The patient interaction can be video-recorded from two perspectives, one capturing the entire thorax and arm postures of the participants, and one capturing the face to determine eye drop instillation success. All data collected from the wearable sensors, eye drop bottle monitor sensors, and video recordings can be synchronized, allowing 4-dimensional movement and positional analysis. These assessments may take approximately 20 minutes.

In some implementations, sensorimotor factors can be quantified. Manipulation of objects such as the eye drop bottle 14 requires proprioceptive awareness of arm position, precise control of fine grasp force, tactile discrimination that codes for object characteristics including surface shape and texture, and dexterous bi-manual hand function. Methods to quantify these abilities are advantageous to help understand factors contributing to the fine sensorimotor control needed to successfully instill eye drop medications.

Upper extremity proprioception (sense of self-movement and body position) can be measured using a limb position reproduction task. While wearing the IMU sensors 52-62 as shown in FIG. 5, for example, patients can be blindfolded and their dominant or non-dominant arm can be passively positioned to different combinations of shoulder abduction/flexion and elbow flexion/extension. The reference positions can be maintained for five seconds, followed by returning the arm to the starting position (arm down to the side of the body). The arm position sense can be examined, requiring vertical positioning of the arm (as would be required when instilling eye drops from a standing or seated position) and horizontal position (associated with eye drop instillation from a supine position). The patient can reproduce the position with the same or opposite arm (matching arm) to identify position sense differences between the dominant and non-dominant arms. Matching with the opposite arm is considered more difficult, as it requires interhemispheric transfer of proprioceptive information. Tasks using perception of both hands and head are important for bimanual coordination when instilling eye drops (i.e., when grasping the eye bottle and holding eyelids). Four trials (or any operable number of trials) can be collected for each arm. Primary measures can include mean end point matching hand position errors and mean variable errors—the latter reflecting consistency of proprioceptive acuity.

To quantify fine grasp force control, patients can be seated and squeeze a hand-held force dynamometer in order to match a force target displayed on a computer screen, for example. The target force may be equivalent to 5% of their maximum grasp force. Once the force target has been reached, patients can be instructed to maintain the force in the target zone for three seconds and then relax. Four trials (or any operable number of trials) can be performed by each hand, with the order counterbalanced across patients. Primary measures can include mean smoothness of force production (i.e., the ability to precisely control hand-related motor recruitment/frequency modulation), and mean force-variability while holding a steady force. The latter is dependent upon monitoring force feedback and compares with efferent muscle force commands.

To quantify tactile discrimination, a custom-designed tactile discrimination device can be used to deliver different spatial patterns to the index finger. Patients can place their finger on a plate containing a 4×6 pin array (1.5 mm pin diameter, 2 mm pin separation) and the pins can then be elevated to create specific patterns on the skin surface. The patterns can be presented for five seconds, at which time the pins can be lowered and the patient can verbally indicate which of four pin patterns shown on a computer screen corresponds to the perceived tactile pattern. The primary measures include mean accuracy in pattern selection and the mean time taken to select a pattern. These measures reflect central processing of tactile feedback related to object manipulation. Four trials (or any operable number of trials) can be recorded for each hand.

To quantify hand function, the Arthritis Hand Function Test can be used. The Arthritis Hand Function Test is a validated functional assessment instrument comprised of subtests that measure manual and applied dexterity and hand strength via maximum grip force. Manual dexterity can be measured using a peg test requiring the placement and removal of nine pegs from a pegboard. Applied dexterity can be quantified by time for the performance of five everyday tasks of fine hand control and bimanual coordination (e.g., tying shoe laces). Maximum grip and pinch (tip and three-point) force can be measured using commercial dynamometers. The primary outcome measures are the mean timed performance related to hand dexterity tasks and maximum hand force. Four trials (or any operable number of trials) can be recorded for each hand and grip force configuration.

The biomechanical parameters and data described herein can also be analyzed, particularly data that objectively define instillation techniques using information collected from the wearable sensors 52-62 and the eye drop bottle monitor 12. The biomechanical parameters are constantly time-varying and include, as described above: 1) elbow flexion-extension and supination-pronation angles, 2) shoulder angle of elevation and plane of elevation, 3) wrist height relative to shoulder, 4) thorax posture (tilt), 5) head posture (tilt), 6) neck flexion-extension and lateral flexion angles, and 7) bottle position (trajectory). In some embodiments, the analysis will focus on the biomechanical parameters at the instant when drops are dispensed. Drop-events (squeezes) are detectable using the eye drop bottle monitor's capacitive sensor 42. Additional information about instillation captured by the IMU 40 includes: the duration of the instillation pause, the steadiness of the bottle during instillation, and the smoothness of the bottle movement. From the biomechanical data, body movements will objectively be described (means, medians, and standard deviations) while instilling drops lying down, sitting, and standing. From video data, the following can be recorded: 1) if the eye drops were successfully instilled into the eye (primary end point), and 2) whether the eye dropper bottle tip touched the ocular or skin surface (“tip contamination,” exploratory end point).

The sensorimotor data can also be analyzed. Measurements of sensorimotor ability are defined as: 1) an assessment of proprioception: absolute matching errors can be calculated by subtracting matching position from the reference position of the hand to determine deficits in proprioception: 2) an assessment of fine grasp force control: the smoothness of force production to the target level can be assessed using the third derivative of the force signal, and hand steadiness can be determined by calculating the coefficient of variation over the three-second force maintenance period: 3) assessment of tactile discrimination: tactile discrimination can be assessed by accuracy and time taken to complete the tasks; and 4) an assessment of hand function: dexterity and maximum hand force can be evaluated by comparing mean timed performance on dexterity tasks and hand force to a normative database. These measures of sensorimotor ability can be compared between the different age groups using analysis of variance. Exploratory analyses can include linear regression models to estimate the association between patient demographics, co-morbid conditions, and baseline physical activity level with sensorimotor measures. Additionally, the relationship between sensorimotor measures and biomechanical measures can be explored with scatter plots, correlation analyses, and regression models.

To predict and determine instillation success, all metrics derived from the sensorimotor and biomechanical data can be included in a machine learning dataset. Scikit-learn can train and compare the classification performance of different supervised machine learning algorithms including Random Forest, Support Vector Machines, Dynamic Time Warping, and Hidden Markov modeling in predicting eye drop instillation success. The Information Gain Attribute in Scikit-learn can identify the worth of features extracted from sensor data, by measuring the information gain with respect to the class and identify the best-fit classification algorithm for the data. The skill scores of the models can be verified using k-folds cross-validation, where the dataset can be trained by k−1 folds and tested on the last fold. An adaptive sampling design can be used where the initial model can be run on the first 10 participants and then run again after each subsequent 10 participants.

To inform the design of the Eye Drop Adherence Monitoring System 10, Scikit-learn can be used to evaluate data from the eye drop bottle monitor sensor platform 24, using the Information Gain Attribute and Gini impurity. This method can determine the minimal sensors and sensorimotor data types required to identify eye drop instillation success with ≥80% accuracy. Similarly, the sensors needed to assess bottle-contamination events can be determined. These results will yield embedded classifiers that predict eye drop instillation and will inform the sensors incorporated into the Eye Drop Adherence Monitoring System 10.

For machine learning, there should be more observations than features analyzed. The eye drop bottle monitor sensors 40, 42, 44 can record high-fidelity, multi-dimensional time series data, resulting in about 11,000 data points for each instillation event. This high-granularity data should enable a robust classification of instillation success. It is expected that >20% of participants will have difficulty instilling eye drops. Therefore, a study recruiting 100 participants can allow ample sampling of different modes of failure, and should provide sufficient observations to construct both training and testing datasets. If algorithms using data only from the eye drop bottle monitor 12 do not have high predictive accuracy for assessing instillation success, metrics derived from the wearable IMU sensors 52-62 or sensorimotor tests can be used to improve the algorithm. Data from an on-patient device may be included to supplement bottle instrumentation, in early testing. The models and training data sets can be used from the biomechanical data analyses to train embedded classifiers on the EAMS on-bottle sensor platform 24, using similar energy-efficient methods as described herein.

The on-bottle sensor platform 24 can be designed to have a minimal profile and advantageously requires modest effort to attach to an eye drop bottle 14. Ideally, this component will be a thin (e.g., less than 3 mm) flexible “sticker” or “sleeve” that attaches to the exterior of existing prescription medication bottles. Advanced flexible printed circuit board (PCB) manufacturing techniques can enable the production of an on-bottle sensor system for low cost when scaled. The on-bottle sensor platform 24 will measure use-events and instillation movements continuously. The data are collected, temporarily stored, and wirelessly transmitted to a base station 28 when within about 10 feet. Participants can be instructed to keep the eye drop monitoring device 12 close to the base station as much as possible to enable frequent data transmission. Since most glaucoma medications are used within twenty-eight days of dispensing, a 30-day operational lifetime per single charge can be targeted, using either a rechargeable battery or supercapacitor. The on-bottle monitor 12 can be recharged on the base station 28 for reuse.

The EAMS 10 can help patients and researchers to: (1) quantify how people interact with their glaucoma medication, (2) inform personalized, scalable approaches to teach eye drop instillation, and (3) develop improved personalized eye drop aids and interventions. It can also be advantageous to improve the sensor system and biomechanical algorithms to broaden the understanding of patient environments, biomechanics, and support systems to inform even more personalized glaucoma self-management support. The EAMS 10 low-cost sensor system can enable large-scale clinical trials to assess the impact of glaucoma self-management support programs on adherence and biological outcomes such as visual field progression. The EAMS 10 can also be used in clinical trials of medications to quantify the impact of adherence on outcomes. Similar systems could also be applied to other complex-to-use medications, such as inhalers and insulin injectors.

It is to be understood that the foregoing description is of one or more preferred example embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “e.g.,” “for instance,” and “such as,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation. In addition, the term “and/or” is to be construed as an inclusive OR. Therefore, for example, the phrase “A, B, and/or C” is to be interpreted as covering all the following: “A”; “B”; “C”; “A and B”; “A and C”; “B and C”; and “A, B, and C.”

1. An eye drop adherence monitoring system, comprising:
a processor; and
a sensor platform configured to attach to an eye drop container, wherein the sensor platform includes one or more sensors configured to measure information relating to one or more instillation movements, wherein the processor is configured to use the information relating to one or more instillation movements to determine instillation success.

2. The eye drop adherence monitoring system of claim 1, wherein the processor and the sensor platform are integrated on a sleeve.

3. The eye drop adherence monitoring system of claim 1, wherein the processor and the sensor platform are integrated on a sticker.

4. The eye drop adherence monitoring system of claim 1, wherein the processor is associated with a base station.

5. The eye drop adherence monitoring system of claim 1, wherein the processor is part of a microcontroller that facilitates wireless communication to a base station.

6. The eye drop adherence monitoring system of claim 1, wherein the information relating to one or more instillation movements includes biomechanical data relating to a posture, a limb position, and/or a dynamic movement of a user.

7. The eye drop adherence monitoring system of claim 6, wherein biomechanical data relating to a posture includes a measurement of thorax tilt, a measurement of head tilt, a measurement of a neck flexion-extension angle and/or a measurement of a neck lateral flexion angle.

8. The eye drop adherence monitoring system of claim 6, wherein biomechanical data relating to a limb position includes a measurement of an elbow flexion-extension angle, a measurement of an elbow supination-pronation angle, a measurement of an angle of elevation for a shoulder, a measurement of a plane of elevation of the shoulder, and a measurement of a wrist height relative to the shoulder.

9. The eye drop adherence monitoring system of claim 1, wherein the information relating to one or more instillation movements includes sensorimotor data relating to a proprioception quantification, a fine grasp force control, a tactile discrimination, and/or a hand function.

10. The eye drop adherence monitoring system of claim 1, wherein the information relating to one or more instillation movements includes a duration of an instillation pause, a steadiness of the eye drop container during instillation, and a smoothness of a position trajectory of the eye drop container.

11. The eye drop adherence monitoring system of claim 1, wherein the one or more sensors includes an inertial measurement unit (IMU), a capacitive sensor, and a magnetic switch, and an ultrasonic transducer.

12. The eye drop adherence monitoring system of claim 1, wherein the processor is configured to calculate a position trajectory for the eye drop container based on an orientation, a velocity, and a position of the eye drop container.

13. The eye drop adherence monitoring system of claim 1, further comprising a radio communication unit integrated with the sensor platform.

14. The eye drop adherence monitoring system of claim 13, wherein the radio communication unit is a backscatter radio communication unit.

15. An adherence monitoring system, comprising:
a processor; and
a sensor platform configured to attach to a container, wherein the sensor platform includes one or more sensors configured to measure information relating to one or more instillation movements, wherein the processor is configured to use the information relating to one or more instillation movements to determine instillation success, wherein the information relating to one or more instillation movements includes biomechanical data relating to a posture, a limb position, and/or a dynamic movement of a user, and wherein the information relating to one or more instillation movements includes sensorimotor data or a duration of an instillation pause, a steadiness of the container during instillation, and a smoothness of a position trajectory of the container.

16. The adherence monitoring system of claim 15, wherein the biomechanical data relating to the posture includes a measurement of thorax tilt, a measurement of head tilt, a measurement of a neck flexion-extension angle and/or a measurement of a neck lateral flexion angle.

17. The adherence monitoring system of claim 15, wherein the biomechanical data relating to the limb position includes a measurement of an elbow flexion-extension angle, a measurement of an elbow supination-pronation angle, a measurement of an angle of elevation for a shoulder, a measurement of a plane of elevation of the shoulder, and a measurement of a wrist height relative to the shoulder.

18. The adherence monitoring system of claim 15, wherein the sensorimotor data relates to a proprioception quantification, a fine grasp force control, a tactile discrimination, and/or a hand function.

19. A method of eye drop adherence monitoring, comprising the steps of:
obtaining information relating to one or more instillation movements from a sensor platform attached to an eye drop container; and
determining instillation success from the information relating to one or more instillation movements.

20. The method of claim 19, wherein the information relating to one or more instillation movements includes biomechanical data relating to a posture, a limb position, and/or a dynamic movement of a user, and wherein the information relating to one or more instillation movements includes sensorimotor data or a duration of an instillation pause, a steadiness of the container during instillation, and a smoothness of a position trajectory of the container.
